(19) United States
(12) Patent Application Publication
(10) Pub. No.: US 2023/0135373 A1
(43) Pub. Date: May 4, 2023

(54) METHODS AND COMPOSITIONS FOR TREATING SLEEP APNEA

(71) Applicant: APNIMED, INC. (DELAWARE), Cambridge, MA (US)

(72) Inventors: Lawrence G. MILLER, Cambridge, MA (US); Ronald FARKAS, Clarksville, MD (US)

(21) Appl. No.: 17/915,153

(22) PCT Filed: Apr. 1, 2021
(86) PCT No.: PCT/US2021/025426

§ 371 Date: Sep. 28, 2022

Related U.S. Application Data
(60) Provisional application No. 63/003,947, filed on Apr. 2, 2020.

(51) Int. Cl. A61K 38/06; A61K 38/05; A61K 45/06; A61K 9/00; A61P 11/04

(52) U.S. Cl. CPC A61K 38/06; A61K 38/05; A61K 45/06; A61K 9/0053; A61P 11/04

(57) ABSTRACT

In general, the invention relates to pharmaceutical compositions comprising a thyrotropin-releasing hormone (TRH) analog, and methods of treating sleep apnea comprising administering a TRH analog.

### CROSS-REFERENCE TO RELATED APPLICATION

This application claims priority to U.S. Provisional Patent Application No. 63/003,947, filed Apr. 2, 2020, the entire contents of which are incorporated herein by reference.

### TECHNICAL FIELD

The present invention provides pharmaceutical compositions comprising a thyrotropin-releasing hormone (TRH) analog, and methods of treating sleep apnea including administering a TRH analog.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of a thyrotropin-releasing hormone (TRH) analog.

Embodiments of this aspect of the invention may include one or more of the following optional features. In some embodiments the TRH analog is selected from the group consisting of, but not limited to, taltirelin, montirelin, JTP-2942, DN-1417, RX-77368, rovatirelin, azetirelin, orotirelin, protirelin, posatirelin, and their pharmaceutically acceptable salts or prodrugs. In some embodiments, the TRH analog is selected from the group consisting of taltirelin and rovatirelin or a pharmaceutically acceptable salt thereof. In some embodiments, the TRH analog (e.g., taltirelin, rovatirelin, or a salt or prodrug of taltirelin or rovatirelin) is in an immediate release formulation, orally disintegrated formulation, intravenous formulation, or oral suspension formulation. In some embodiments, the TRH analog (e.g., taltirelin, rovatirelin, or a salt or prodrug of taltirelin or rovatirelin) is in an extended release formulation. In some embodiments, the TRH analog (e.g., taltirelin, rovatirelin, or a salt or prodrug of taltirelin or rovatirelin) is present in an amount of from about 0.1 mg to about 50 mg. For example, the TRH analog (e.g., taltirelin, rovatirelin, or a salt or prodrug of taltirelin or rovatirelin) may be in an immediate release formulation and may be present in an amount of from about 0.1 mg to about 25 mg. Or for example, the TRH analog (e.g., taltirelin, rovatirelin, or a salt or prodrug of taltirelin or rovatirelin) may be in an extended release formulation and may be present in an amount of from about 0.5 mg to about 50 mg. In some embodiments, the method is a monotherapy of the TRH analog (e.g., taltirelin, rovatirelin, or a salt or prodrug of taltirelin or rovatirelin). In some embodiments, the method further comprises administering (e.g., concomitantly administering) an effective amount of one or more additional active agents either separately or in a fixed dosage formulation. In some embodiments, the condition associated with pharyngeal airway collapse is sleep apnea or simple snoring. In some embodiments, the condition associated with pharyngeal airway collapse is obstructive sleep apnea (OSA). In some embodiments, the subject is in a non-fully conscious state (e.g., sleep). In some embodiments, the TRH analog (e.g., taltirelin, rovatirelin, or a salt or prodrug of taltirelin or rovatirelin) and the one or more additional active agents are administered in a single composition. In some embodiments, the single composition is an oral administration form (e.g., a syrup, slurry, pill, tablet, troche, capsule, or patch). In some embodiments, the TRH analog is taltirelin or a pharmaceutically acceptable salt thereof. In some embodiments, the taltirelin is administered once daily at a dose of from about 5 mg to about 10 mg.

Another aspect of the present invention provides a pharmaceutical composition comprising a TRH analog and a pharmaceutically acceptable excipient or carrier.

Embodiments of this aspect of the invention may include one or more of the following optional features. In some embodiments the TRH analog is selected from the group consisting of, but not limited to, taltirelin, montirelin, JTP-2942, DN-1417, RX-77368, rovatirelin, azetirelin, orotirelin, protirelin, posatirelin, and their pharmaceutically acceptable salts or prodrugs. In some embodiments, the TRH analog is selected from the group consisting of taltirelin and rovatirelin or a pharmaceutically acceptable salt thereof. In some embodiments, the TRH analog (e.g., taltirelin, rovatirelin, or a salt or prodrug of taltirelin or rovatirelin) is in an immediate release formulation. In some embodiments, the TRH analog (e.g., taltirelin, rovatirelin, or a salt or prodrug of taltirelin or rovatirelin) is in an extended release formulation. In some embodiments, the TRH analog (e.g., taltirelin, rovatirelin, or a salt or prodrug of taltirelin or rovatirelin) is present in an amount of from about 0.1 to about 50 mg. For example, the TRH analog (e.g., taltirelin, rovatirelin, or a salt or prodrug of taltirelin or rovatirelin) may be in an immediate release formulation and may be present in an amount of from about 0.1 mg to about 25 mg. Or for example, the TRH analog (e.g., taltirelin, rovatirelin, or a salt or prodrug of taltirelin or rovatirelin) may be in an extended release formulation and may be present in an amount of from about 0.5 mg to about 50 mg. In some embodiments, the TRH analog (e.g., taltirelin, rovatirelin, or a salt or prodrug of taltirelin or rovatirelin) and one or more additional active agents are disposed together in a pharmaceutically acceptable carrier to form a single dosage agent. In some embodiments, the TRH analog (e.g., taltirelin, rovatirelin, or a salt or prodrug of taltirelin or rovatirelin) is the sole active agent in a pharmaceutical composition. In some embodiments, the TRH analog (e.g., taltirelin, rovatirelin, or a salt or prodrug of taltirelin or rovatirelin) and one or more additional active agents are separately disposed in pharmaceutically acceptable carriers to form a separate dosing agent. In some embodiments, the composition is for use in treating a subject having a condition associated with pharyngeal airway collapse. In some embodiments, the condition associated with pharyngeal airway collapse is sleep apnea or simple snoring. In some embodiments, the condition associated with pharyngeal airway collapse is OSA. In some embodiments, the subject is in a non-fully conscious state (e.g., sleep).

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGURES, and from the claims.

### BRIEF DESCRIPTION OF THE DRAWINGS

The following FIGURES are provided by way of example and are not intended to limit the scope of the claimed invention.

FIG. 1. Graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect contraction of respiratory muscles. The last channel indicates oxyhemoglobin saturation.

### DETAILED DESCRIPTION

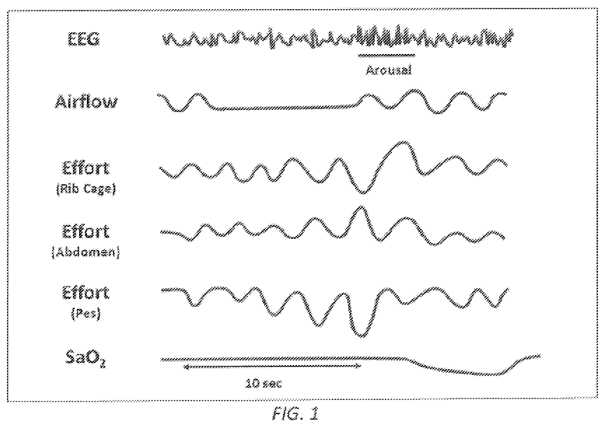

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland, W R. et al., The new AASM criteria for scoring hypopneas: Impact on the apnea hypopnea index, SLEEP 2009, 32:150-157).

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young, T. et al., Burden of sleep apnea: rationale, design, and major findings of the Wisconsin sleep cohort study, WMJ 2009, 108:246-9; Peppard, P E. et al., Increased prevalence of sleep-disordered breathing in adults, Am J Epidemiol 2013, 177:1006-14.)

The present leading treatment is continuous positive airway pressure (CPAP). CPAP is effective in virtually all patients, and approximately 85% of diagnosed patients are treated, but compliance is low. Patients find CPAP uncomfortable and often intolerable; at least 30% of patients (up to 80%) are regularly non-adherent and thus untreated (Weaver, TE. and Grunstein RR. Adherence to continuous positive airway pressure therapy: the challenge to effective treatment, Proc Am Thorac Soc. 2008, 5:173-178). Other treatment modalities with variable rates of success include oral appliances (10%) and surgery (5%), but neither is likely to be effective across the general population. No pharmacologic treatments have been shown to be effective to date.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Hanzel, D A et al., Response of obstructive sleep apnea to fluoxetine and protriptyline, Chest. 1991, 100:416-21; Brownell, L G. et al., Protriptyline in obstructive sleep apnea: a double-blind trial, N Engl J Med 1982, 307:1037-1042; Sangal, R B. et al., Atomoxetine improves sleepiness and global severity of illness but not the respiratory disturbance index in mild to moderate obstructive sleep apnea with sleepiness, Sleep Med. 2008, 9:506-10; Marshall, N S. et al., Two randomized placebo-controlled trials to evaluate the efficacy and tolerability or mirtazapine for the treatment of obstructive sleep apnea, Sleep 2008, 31:824-31; Eckert, D J. et al., Eszopiclone increases the respiratory arousal threshold and lowers the apnoea/hypopnea index in obstructive sleep apnoea patients with a low arousal threshold, Clin Sci (Lond). 2011, 120):505-14; Taranto-Montemurro, L. et al., Effects of tiagabine on slow wave sleep and arousal threshold in patients with obstructive sleep apnea, Sleep. 2017 Feb. 1;40(2).

Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). Generally, the methods include administering a therapeutically effective amount of a thyrotropin-releasing hormone analog (a “TRH analog”), to a subject who is in need of, or who has been determined to be in need of, such treatment. In some embodiments, the TRH analog is selected from the group consisting of taltirelin, montirelin, JTP-2942, DN-1417, RX-77368, rovatirelin, azetirelin, orotirelin, protirelin, posatirelin, and their pharmaceutically acceptable salts or prodrugs. In some embodiments, the TRH analog is selected from the group consisting of taltirelin and rovatirelin or a pharmaceutically acceptable salt thereof. In some embodiments, the TRH analog (e.g., taltirelin, rovatirelin, or a salt or prodrug of taltirelin or rovatirelin) is administered as a monotherapy. In some embodiments, the TRH analog (e.g., taltirelin, rovatirelin or a salt or prodrug of taltirelin or rovatirelin) is administered in combination with one or more additional active agents (e.g., two or more, or three or more, additional active agents).

In some embodiments, the one or more additional active agents comprises a norepinephrine reuptake inhibitor. In some embodiments, the norepinephrine reuptake inhibitor is a norepinephrine selective reuptake inhibitor (NSRI), e.g., selected from the group consisting of amedalin, atomoxetine, CP-39,332, daledalin, edivoxetine, esreboxetine, lortalamine, nisoxetine, reboxetine, talopram, talsupram, tandamine, viloxazine and combinations thereof or a pharmaceutically acceptable salt thereof. In some embodiments, the norepinephrine reuptake inhibitor is selected from atomoxetine, reboxetine, 4-hydroxyatomoxetine, or a combination thereof or a pharmaceutically acceptable salt thereof. In some embodiments, the norepinephrine reuptake inhibitor is a norepinephrine non-selective reuptake inhibitor (NNRI), e.g., selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, protryptyline, radafaxine, tapentadol, teniloxazine, venlafaxine, and combinations thereof or a pharmaceutically acceptable salt thereof. In some embodiments, the norepinephrine reuptake inhibitor is atomoxetine or a pharmaceutically acceptable salt thereof, and in specific embodiments, the dosage of atomoxetine is from about 20 to about 200 mg, e.g., about 25 to about 100 mg (e.g., 25 mg, 50 mg, 75 mg, or 80 mg). In some embodiments, the norepinephrine reuptake inhibitor is 4-hydroxyatomoxetine or a pharmaceutically acceptable salt thereof, and in specific embodiments, the dosage of 4-hydroxyatomoxetine is from about 20 to about 200 mg, e.g. about 25 to about 100 mg (e.g., 25 mg, 50 mg, 75 mg, or 80 mg).

In some embodiments, the one or more additional active agents comprises a muscarinic receptor antagonist. In some embodiments, the muscarinic receptor antagonist is an M2 receptor agonist, e.g., selected from the group consisting of atropine, propantheline, bethanechol, solifenacin, darifenacin, tolterodine, fesoterodine, trospium, and oxybutynin or a pharmaceutically acceptable salt thereof. In some embodiments, the muscarinic receptor antagonist is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl and trihexyphenidyl or a pharmaceutically acceptable salt thereof. In some embodiments, the muscarinic receptor antagonist is oxybutynin, e.g., (R)-oxybutynin, or a pharmaceutically acceptable salt thereof. In some embodiments, the muscarinic receptor antagonist is oxybutynin or a pharmaceutically acceptable salt thereof, and in specific embodiments, the dosage of oxybutynin is from about 1 to about 15 mg (e.g., 2-10 mg, or 1 mg, 2 mg, 3 mg, 4 mg, 5 mg, or 6 mg). In some embodiments, the muscarinic receptor antagonist is (R)-oxybutynin or a pharmaceutically acceptable salt thereof, and in specific embodiments, the dosage of (R)-oxybutynin is from about 1 to about 15 mg (e.g., 1-5 mg, or 1 mg, 2 mg, 3 mg, 4 mg, 5 mg, or 6 mg).

In some embodiments, the one or more additional active agents comprises a hypnotic, e.g., a hypnotic selected from the group consisting of zolpidem, zopiclone, eszopiclone, trazodone, zaleplon, benzodiazepines, gabapentin, tiagabine, and xyrem or a pharmaceutically acceptable salt thereof.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA will result in decreased AHI.

In general, an “effective amount” of a compound refers to an amount sufficient to elicit the desired biological response, e.g., to treat a condition associated with pharyngeal airway collapse, e.g., to treat sleep apnea or snoring.

An effective amount can be administered in one or more administrations, applications or dosages. The compositions can be administered from one or more times per day to one or more times per week; including once every other day. In some embodiments, the compositions are administered daily. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Taltirelin is the generic name of the pharmaceutical substance with the chemical name (4S)-N-[2S)-1-[(2S)-2-carbamoylpyrrolidin-1-yl]-3-(1H-imidazol-5-yl)-1-oxopropan-2-yl]-1-methyl-2,6-dioxo-1,3-diazinane-4-carboxamide, and stereoisomers, hydrates and salts thereof. Taltirelin is commercially available in Japan as taltirelin hydrate (Ceredist®).

Atomoxetine is the generic name of the pharmaceutical substance with the chemical name (-)-N-Methyl-3-phenyl-3-(o-tolyloxy)-propylamine, and its pharmaceutical salts. Atomoxetine is the R(-) isomer as determined by x-ray diffraction. In some embodiments, atomoxetine may be atomoxetine hydrochloride.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S- enantiomers, or an isolated enantiomer, e.g., the R-enantiomer. In various embodiments, oxybutynin may be oxybutynin chloride or (R)-oxybutynin chloride.

In some embodiments, the methods include administering a dose of from about 0.1 mg to about 50 mg of a TRH analog (e.g., taltirelin, rovatirelin, or a salt or prodrug of taltirelin or rovatirelin). In some embodiments, the methods include administering a dose of from about 0.5 mg to about 25 mg of a TRH analog (e.g., taltirelin or rovatirelin, or a salt or prodrug of taltirelin or rovatirelin). In some embodiments, the methods include administering a dose of from about 1 mg to about 25 mg of a TRH analog (e.g., taltirelin, rovatirelin, or a salt or prodrug of taltirelin or rovatirelin). In some embodiments, the methods include administering a dose of from about 0.1 mg to about 10 mg of a TRH analog (e.g., taltirelin or rovatirelin, or a salt or prodrug of taltirelin or rovatirelin). In some embodiments, the methods include administering a dose of from about 0.5 mg to about 15 mg of a TRH analog (e.g., taltirelin or rovatirelin, or a salt or prodrug of taltirelin or rovatirelin). In some embodiments, the methods include administering a dose of from about 5 mg to about 25 mg of a TRH analog (e.g., taltirelin, rovatirelin, or a salt or prodrug of taltirelin or rovatirelin). In some embodiments, the methods include administering a dose of about 0.5, 1, 2, 2.5, 3, 4, 5, 6, 7, 7.5, 8, 9, 10, 11, 12, 13, 14, 15, 16, 17, 18, 19, 20, 25, 30, 35, 40, 45 or 50 mg of a TRH analog (e.g., taltirelin or rovatirelin, or a salt or prodrug of taltirelin or rovatirelin). In some embodiments, the dose is a daily dose.

In some embodiments, the methods include administering a dose of from about 1 mg to about 20 mg of taltirelin or a pharmaceutically acceptable salt thereof. In some embodiments, the methods include administering a dose of from about 5 mg to about 10 mg of taltirelin or a pharmaceutically acceptable salt thereof (e.g., 5 mg, 7.5 mg, or 10 mg).

Provided herein is a method of treating a subject having a condition associated with pharyngeal airway collapse (e.g., sleep apnea or OSA), the method comprising administering to a subject in need thereof an effective amount of taltirelin or a pharmaceutically acceptable salt thereof.

Also provided herein is a method of treating a subject having a condition associated with pharyngeal airway collapse (e.g., sleep apnea or OSA), the method comprising administering to a subject in need thereof a once daily dose of from about 5 mg to about 10 mg of taltirelin or a pharmaceutically acceptable salt thereof.

In some embodiments, the TRH analog (e.g., taltirelin, rovatirelin, or a salt or prodrug of taltirelin or rovatirelin) may be in an immediate release formulation. For example, the immediate release formulation may comprise a TRH analog (e.g., taltirelin, rovatirelin, or a salt or prodrug of taltirelin or rovatirelin) in an amount of from about 0.1 mg to about 25 mg, e.g., from about 0.5 mg to about 10 mg; or from about 5 to about 25 mg; or about 0.5, 1, 2, 2.5, 3, 4, 5, 6, 7, 7.5, 8, 9, 10, 11, 12, 13, 14, 15, 16, 17, 18, 19, 20 or 25 mg.

In some embodiments, the TRH analog (e.g., taltirelin, rovatirelin, or a salt or prodrug of taltirelin or rovatirelin) may be in an extended release formulation. For example, the extended release formulation may comprise a TRH analog (e.g., taltirelin, rovatirelin, or a salt or prodrug of taltirelin or rovatirelin) in an amount of from about 0.5 mg to about 50 mg, e.g., from about 1 mg to about 15 mg; or from about 5 to about 50 mg; or about 0.5, 1, 2, 2.5, 3, 4, 5, 6, 7, 7.5, 8, 9, 10, 11, 12, 13, 14, 15, 16, 17, 18, 19, 20, 25, 30, 35, 40, 45 or 50 mg.

In some embodiments the TRH analog is selected from the group consisting of, but not limited to, taltirelin, montirelin, JTP-2942, DN-1417, RX-77368, rovatirelin, azetirelin, orotirelin, protirelin, posatirelin, and their pharmaceutically acceptable salts or prodrugs.

In some embodiments, the TRH analog is taltirelin or a pharmaceutically acceptable salt thereof.

In some embodiments, the TRH analog is rovatirelin or a pharmaceutically acceptable salt thereof.

Pharmaceutical Compositions

The methods described herein include the use of pharmaceutical compositions comprising a TRH analog. In some embodiments the TRH analog is selected from the group consisting of, but not limited to, taltirelin, montirelin, JTP-2942, DN-1417, RX-77368, rovatirelin, azetirelin, orotirelin, protirelin, posatirelin, and their pharmaceutically acceptable salts or prodrugs. In some embodiments, the TRH analog is selected from the group consisting of taltirelin and rovatirelin or a pharmaceutically acceptable salt thereof. In some embodiments, the TRH analog is taltirelin or a pharmaceutically acceptable salt thereof. In some embodiments, the taltirelin or pharmaceutically acceptable salt thereof is present in the pharmaceutical composition in an amount of from about 1 mg to about 20 mg, or from about 5 mg to about 10 mg, e.g., 5 mg, 7.5 mg or 10 mg.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., hypnotics including zolpidem, zopiclone, eszopiclone, trazodone, zaleplon, benzodiazepines, gabapentin, tiagabine, and xyrem or a pharmaceutically acceptable salt thereof. In such embodiments where patients have a low arousal threshold, a hypnotic can be used as a supplementary active compound to increase the arousal threshold of the patient having OSA, pharyngeal airway collapse, or a combination thereof. In some embodiments, the arousal threshold of a patient can be measured by polysomnography (PSG). In some embodiments, the pharmaceutical composition comprises a TRH analog (e.g., taltirelin, rovatirelin, or a salt or prodrug of taltirelin or rovatirelin) and a hypnotic. In some embodiments, the method of treating a subject having a condition associated with pharyngeal airway collapse comprises administering to a subject in need thereof an effective amount of (i) a TRH analog (e.g., taltirelin, rovatirelin, or a salt or prodrug of taltirelin or rovatirelin), and (ii) a hypnotic.

Pharmaceutical compositions are typically formulated to be compatible with its intended route of administration. Examples of routes of administration include systemic oral, transdermal administration, and parenteral administration.

Methods of formulating suitable pharmaceutical compositions using pharmaceutically acceptable carriers are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, a TRH analog (e.g., taltirelin, rovatirelin, or a salt or prodrug of taltirelin or rovatirelin) and one or more additional active agents are disposed together in one or more pharmaceutically acceptable carriers to form a single dosage agent. The single dosage agent comprises the TRH analog (e.g., taltirelin, rovatirelin, or a salt or prodrug of taltirelin or rovatirelin), the one or more additional active agents, and the pharmaceutically acceptable carrier. In other embodiments, a TRH analog (e.g., taltirelin, rovatirelin, or a salt or prodrug of taltirelin or rovatirelin) and one or more additional active agents are separately disposed in one or more pharmaceutically acceptable carriers to form separate dosing agents. The separate dosing agents may be used in any combination to provide treatment to a subject having a condition associated with pharyngeal airway collapse, sleep apnea, or a combination thereof.

Systemic administration of a TRH analog (e.g., taltirelin, rovatirelin, or a salt or prodrug of taltirelin or rovatirelin) can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn, J A. et al., An update on the use of transdermal oxybutynin in the management of overactive bladder disorder, Ther Adv Urol. 2016, Apr.; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

Administration of a TRH analog (e.g., taltirelin, rovatirelin, or a salt or prodrug of taltirelin or rovatirelin) can also be by parenteral, e.g., intravenous, means.

The pharmaceutical compositions can be included in a container, pack, or dispenser together with instructions for administration or use in a method described herein.

In some embodiments, the pharmaceutical composition is for use in treating a condition associated with pharyngeal airway collapse. In some embodiments, the condition is sleep apnea (e.g., OSA) or snoring (e.g., simple snoring). In certain embodiments, provided herein is a pharmaceutical composition comprising atomoxetine or a pharmaceutically acceptable salt thereof and spironolactone or a pharmaceutically acceptable salt thereof, and optionally oxybutynin (e.g., (R)-oxybutynin) or a pharmaceutically acceptable salt thereof for use in treating sleep apnea (e.g., OSA) or snoring (e.g., simple snoring).

### Examples

The invention is further described in the following examples, which do not limit the scope of the invention described in the claims.

### Example 1. Pilot Study

In healthy human individuals, the effect of once daily taltirelin 5 mg on genioglossus muscle activity is measured in a pilot study.

A first group of the patients is given once daily taltirelin 5 mg. A second group of patients is given placebo. Genioglossus muscle activity (EMG_{GG}, quantified as a percentage of maximum) is measured during quiet wakefulness. Each peak EMG_{GG} of a single breath is measured and is plotted against the corresponding epiglottic pressure. In addition, EMG_{GG} is measured during stable NREM sleep.

It is expected that there will be a variable but clear reduction in EMG_{GG} activity during sleep on the placebo night and that, in contrast, when patients are administered taltirelin, the sleep-related reduction in pharyngeal muscle activity will be partially or completely prevented.

It is expected that, compared to placebo, the tested drug will yield a much higher EMG_{GG} activity during NREM sleep. It is also expected that the drug will be effective during REM sleep for those subjects exhibiting REM sleep when administered the tested drug.

### Example 2. Crossover Study

A placebo-controlled, double-blinded, randomized, crossover trial in OSA human patients is performed. Participants receive treatment (once daily taltirelin 5-10 mg) or placebo in randomized order 30 minutes before sleep. The study drug taltirelin is expected to reduce the apnea hypopnea index and all patients are expected to experience an improvement in OSA severity. Additional benefits expected are increased genioglossus muscle responsiveness to an increase in ventilatory drive, improved upper airway muscle activity, improved ventilation, increased oxygen levels (SaO₂), increased total sleep time and improved sleep efficiency.

### Example 3. Crossover Study in Japan

A randomized, double-blind, crossover comparative study of the efficacy of taltirelin in obstructive sleep apnea is performed. Participants receive treatment—taltirelin 7.5 mg (oral)—or placebo once daily 30 minutes before bedtime. After registration, the drug priority group will take the drug treatment 30 minutes before bed on the day of the first PSG, and will take the placebo 30 minutes before bed on the day of the second PSG 1-2 weeks later. The placebo priority group will take the placebo 30 minutes before bed on the day of the first PSG, and will take the drug treatment 30 minutes before bed on the day of the second PSG 1-2 weeks later.

The approved dosage and administration of taltirelin is 5 mg twice a day, administered on consecutive days. In order to verify the efficacy of taltirelin for obstructive sleep apnea, we investigated using the maximum dose, taking safety into account. In a test of administration of a single oral dose (0.5 mg-40 mg) to Japanese subjects, although no safety events of note were observed for single doses of 10 mg and higher, headache and nausea were reported, and since there was concern about the effect of these symptoms on sleep, the dosage possibilities were either 5 mg and 7.5 mg, and the maximum of a single dose administration of 7.5 mg was selected.

The following items are evaluated in the study.

Main Evaluation Item (Primary endpoint): AHI measured according to the American Academy of Sleep Medicine rules.

Secondary Evaluation Items (Secondary endpoints):
AHI by alternative definitions of hypopnea (4% or greater decrease in arterial oxygen saturation and 30% or greater decrease in airflow)
Hypoxia-ischemia (area under the curve when arterial oxygen saturation is reduced×number of events)
Arousal index (number of times awakened/hours of sleep)
Sleep efficiency (total hours of sleep/hours in bed)
Visual evaluation scale to evaluate quality of sleep
Karolinska Sleepiness Scale
Epworth Sleepiness Scale.

Safety Evaluation Items: Blood pressure (systolic and diastolic) and diseases, etc. and side effects (abnormal thyroid hormone levels, etc.) occurring in research subjects.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject having a condition associated with pharyngeal airway collapse, the method comprising administering to a subject in need thereof an effective amount of a thyrotropin-releasing hormone (TRH) analog selected from the group consisting of taltirelin, montirelin, JTP-2942, DN-1417, RX-77368, rovatirelin, azetirelin, orotirelin, protirelin, posatirelin, and their pharmaceutically acceptable salts or prodrugs.

2. The method of claim 1, comprising administering to the subject a daily dose of from about 0.1 mg to about 50 mg of the TRH analog.

3. The method of claim 1, comprising administering to the subject a daily dose of from about 0.5 mg to about 15 mg of the TRH analog.

4. The method of claim 1, wherein the TRH analog is administered orally.

5. The method of claim 1, wherein the TRH analog is in an immediate release formulation.

6. The method of claim 1, wherein the TRH analog is in an extended release formulation.

7. The method of claim 1, wherein the TRH analog is in an immediate release formulation and is administered at a dosage of from about 0.5 to about 10 mg.

8. The method of claim 1, wherein the TRH analog is in an extended release formulation and is administered at a dosage of from about 1 to about 15 mg.

9. The method of claim 1, wherein the method further comprises administering to the subject in need thereof an effective amount of one or more additional active agents selected from a norepinephrine reuptake inhibitor, a muscarinic receptor antagonist, or both.

10. The method of claim 1, wherein the method is a monotherapy of the TRH analog.

11. The method of claim 1, wherein the method further comprises administering to the subject in need thereof an effective amount of a hypnotic selected from the group consisting of zolpidem, zopiclone, eszopiclone, trazodone, zaleplon, benzodiazepines, gabapentin, tiagabine, and xyrem or a pharmaceutically acceptable salt thereof.

12. The method of claim 1, wherein the TRH analog is selected from the group consisting of taltirelin and rovatirelin or a pharmaceutically acceptable salt thereof.

13. The method of claim 1, wherein the TRH analog is taltirelin or a pharmaceutically acceptable salt thereof.

14. The method of claim 1, wherein the TRH analog is rovatirelin or a pharmaceutically acceptable salt thereof.

15. The method of claim 1, wherein the condition associated with pharyngeal airway collapse is sleep apnea or snoring.

16. The method of claim 1, wherein the condition associated with pharyngeal airway collapse is obstructive sleep apnea (OSA).

17. The method of claim 1, wherein the subject is in a non-fully conscious state such as sleep.

18. The method of claim 13, wherein the taltirelin or pharmaceutically acceptable salt thereof is administered at a dose of from about 5 mg to about 10 mg once daily.

19. The method of claim 1, wherein the TRH analog is administered orally in an oral administration form selected from a syrup, slurry, pill, tablet, troche, capsule, or patch.

20.-31. (canceled)

32. The method of claim 18, wherein the condition associated with pharyngeal airway collapse is obstructive sleep apnea (OSA).
